(19) United States
(12) Patent Application Publication
(10) Pub. No.: US 2011/0276102 A1
(43) Pub. Date: Nov. 10, 2011

(54) REDUNDANT PACING SYSTEM WITH LEADED AND LEADLESS PACING

(76) Inventor: Todd J. Cohen, Mineola, NY (US)

(21) Appl. No.: 13/101,958

(22) Filed: May 5, 2011

Related U.S. Application Data
(60) Provisional application No. 61/331,669, filed on May 5, 2010.

(51) Int. Cl. A61N 1/365; A61N 1/362; A61N 1/39; A61N 1/05

(52) U.S. Cl. 607/4; 607/119; 607/32; 607/29; 607/17

(57) ABSTRACT

A pacing system includes a controller operable to provide control signals indicating desired pacing signals, a pulse generator connected to the controller and operable to receive the control signals and to generate the desired pacing signals based on the control signals, at least one lead electrically connected to the pulse generator and extending into a user's heart and operable to provide the pacing signals to the heart, at least one electrode positioned in the user's heart and electrically connected to the at least one lead, the at least one electrode in contact with the user's heart and operable to stimulate the heart based on the pacing signals; and a transceiver, in communication with the pulse generator and operable to selectively transmit the pacing signals to the electrode wirelessly. The transceiver is controlled by the controller to transmit the pacing signals when pacing signals are not received by the electrode from the at least one lead. The lead may include multiple leads held together in a sugar moiety as a unitary body for insertion into the heart. Once in the heart, the sugar moiety dissolves to allow the leads to separate for implantation at different points in the heart.

### CROSS-REFERENCE TO RELATED APPLICATIONS

The present application claims benefit of and priority to U.S. Provisional Patent Application Ser. No. 61/331,669 filed May 5, 2010 entitled VENTRICULAR PACING REDUNDANCY FOR PACEMAKER DEPENDENT PATIENTS, the entire content of which is hereby incorporate by reference herein.

### BACKGROUND

1. Field of the Disclosure

The present disclosure relates to a pacing system that provides redundant pacing and also allows for pacing signals to be transmitted via leads or wirelessly.

2. Related Art

Approximately 5-10 percent of all pacemaker systems are implanted in patients who have a significant pacing requirement. That is, these patients are either completely dependent on the pacemaker, or would suffer negative symptoms if pacing to the ventricle stopped. These symptoms include, but are not limited to, hypotension, lightheadedness, dizziness (presyncope), syncope and even death.

Unfortunately, there are many reasons why a pacing device might fail to provide required pacing signals. Failure may occur at any of several links in a chain of elements that make up the device. Most simply, a pacing device will include a power source, generally a battery that powers hardware in the device to provide the pacing signals. The hardware is generally controlled by a controller, typically in the form of instructions provided in appropriate software executed by a microprocessor or other suitable control device. Pacing and sensing functions are generally provided via a lead, which is attached to a pulse generator of the device. The pulse generator, hardware, software and power source are generally incorporated in a single element or housing. The lead typically extends out of the housing and into the user's heart.

The lead will generally include at least one conductor connected to an electrode positioned in the heart. Failures may occur in or between any of these elements that may result in no pacing signals being provided to the patient's heart. The lead is the element most prone to failure. Faults may occur in the connection of the lead to the pulse generator, in the lead itself or in the connection of the lead to the electrode.

For those patients who are dependent on pacing, any of these faults could be deadly. Accordingly, it would be beneficial to provide a pacing system that maintains constant pacing despite certain failures.

### SUMMARY

It is an object of the present invention to provide a pacing system that provides redundancy and pacing signals via leads and wirelessly.

A lead structure for use in a redundant pacing system in accordance with the an embodiment of the present disclosure includes a first lead element including at least one conductor connected to a first terminal of a pulse generator of the pacing system, a second lead element including at least a second conductor and connected to a second terminal of the pulse generator of the pacing system, the first lead element and the second lead element held together via a sugar moiety for a predetermined period of time in a user's body.

A pacing system in accordance with an embodiment of the present disclosure includes a controller operable to provide control signals indicating desired pacing signals for use in stimulating a user's heart, a pulse generator connected to the controller and operable to receive the control signals and to generate the desired pacing signals based on the control signals, at least one lead electrically connected to the pulse generator and extending into the user's heart and operable to provide the pacing signals to the user's heart, at least one electrode positioned in the user's heart and electrically connected to the at least one lead, the at least one electrode in contact with the user's heart and operable to stimulate the heart based on the pacing signals and a transceiver, in communication with the pulse generator and operable to selectively transmit the pacing signals to the electrode wirelessly. The transceiver is controlled by the controller to transmit the pacing signals when pacing signals are not received by the electrode from the at least one lead.

A pacing system in accordance with another embodiment of the present application includes a controller operable to provide control signals indicating desired pacing signals to stimulate a user's heart, a pulse generator connected to the controller and operable to receive the control signals and to generate the desired pacing signals based on the control signals, at least one lead electrically connected to the pulse generator and extending into the user's heart and operable to provide the pacing signals to the user's heart, at least a first electrode positioned in the user's heart and electrically connected to the at least one lead, the first electrode in contact with the user's heart and operable to stimulate the heart based on the pacing signals, a transceiver, in communication with the pulse generator and operable to selectively transmit the pacing signals wirelessly and a second electrode separate from the lead and positioned in the user's heart, the second electrode including a receiving circuit operable to receive the wireless pacing signals and operable to stimulate the user's heart based on the received wireless pacing signals. The transceiver is controlled by the controller to wirelessly transmit the pacing signals when pacing signals are not received by the first electrode from the at least one lead.

A pacing system in accordance with an embodiment of the present application includes a housing configured for positioning in a user's heart; a controller, mounted in the housing and operable to provide control signals indicating desired pacing signals for use in stimulating the user's heart; a pulse generator, mounted in the housing and connected to the controller and operable to receive the control signals and to generate the desired pacing signals based on the control signals; at least a first electrode, mounted in the housing and electrically connected to the pulse generator, the first electrode in contact with the user's heart and operable to stimulate the heart based on the pacing signals; and a fastener configured and operable to attach the housing to the user's heart such that the electrode is in contact with the user's heart.

A pacing system in accordance with an embodiment of the present application includes a controller operable to provide control signals indicating desired pacing signals for use in stimulating a user's heart; a pulse generator connected to the controller and operable to receive the control signals and to generate the desired pacing signals based on the control signals; a first lead electrically connected to the pulse generator and extending into the user's heart to a first position; a first electrode positioned in the user's heart at the first position and electrically connected to the first lead, the first electrode configured and operable to stimulate the user's heart based on the pacing signals from the pulse generator and to sense activity in the first position in the user's heart and to provide first sensed information regarding the activity in the first position to the pulse generator and controller; a second lead electrically connected to the pulse generator and extending into the user's heart to a second position; and a second electrode positioned in the user's heart at the second position and electrically connected to the second lead, the second electrode configured and operable to stimulate the user's heart based on the pacing signals and to sense activity at the second position in the user's heart and to provide second sensed information regarding the activity at the second position to the pulse generator. The controller controls the pulse generator to provide pacing signals to the first electrode via the first lead for a period of time and to provide pacing signals to the second electrode via the second lead when the first sensed information indicates a fault in one of the first lead and the first electrode.

Other features and advantages of the present invention will become apparent from the following description of the invention which refers to the accompanying drawings.

### BRIEF DESCRIPTION OF THE DRAWINGS

FIG. 1 is an exemplary block diagram illustrating a pacing system in accordance with an embodiment of the present application.

FIG. 2 is an exemplary illustration of a cross-section of a lead for use in the pacing system in accordance with an embodiment of the present application.

FIG. 3 is another view of the lead of FIG. 2.

FIG. 4 is an exemplary block diagram illustrating a pacing system in accordance with another embodiment of the present application.

FIG. 5 is an illustration of an exemplary embodiment of a bipolar electrode.

FIG. 6 is an exemplary embodiment of a lead with an electrode positioned on the distal end thereof.

FIG. 7A illustrates a detailed view of an exemplary electrode including receiving circuit for receiving wireless signals in accordance with an embodiment of the present application.

FIG. 7B illustrates the exemplary electrode of FIG. 7A with a coiled antenna compressed as it is attached to the heart.

FIG. 7C illustrates the exemplary electrode of FIGS. 7A-7B connected to the user's heart.

FIG. 8 illustrates an alternative embodiment of a pacing system in accordance with an embodiment of the present application.

### DETAILED DESCRIPTION OF THE EMBODIMENTS

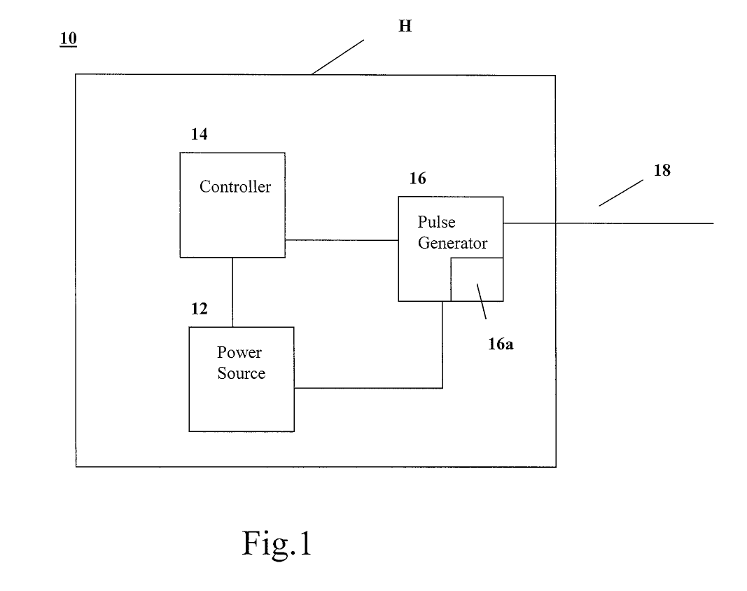

In a preferred embodiment, illustrated generally in the exemplary block diagram of FIG. 1, the pacing device 10 of the present application includes a power source 12, a controller 14 and a pulse generator 16. The pulse generator 16 includes terminals that are connected to the lead 18, which provides the pacing signals from the pulse generator to the user's heart. In addition, the lead 18 may be used to convey information from the heart to the controller 14, via the pulse generator 16, for example. The controller 14 may use this information in controlling the pulse generator 16. The system 10 of FIG. 1, however also provides for a second mode of operation where at least pacing information is transmitted wirelessly to the heart such that pacing will occur even where there is a fault in the lead 18, or the connection between the lead and the pulse generator 16. For this purpose, the pulse generator 16 preferably also includes a transceiver 16a (transmitter/receiver) that is operable to selectively transmit pacing information wirelessly.

The controller 14 may be a microprocessor or any suitable control device. The controller 14 will typically include, or be connected to a memory device (not shown). The memory device preferably stores a series of instructions regarding operation of the pacing system 10 to maintain appropriate pacing for the user. While the controller 14 and pulse generator 16 are illustrated as separate devices, they may be combined together if desired. Further, the transceiver 16a may be combined with the pulse generator 16, the controller 14 or may be embodied as a separate element.

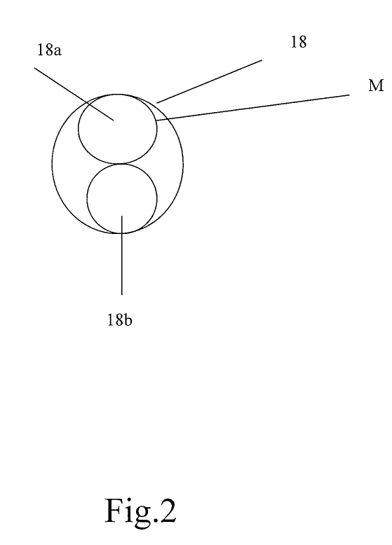

The lead structure, or lead, 18 in FIG. 2 is shown as a single wire, however, may include multiple conductors, if desired, for example, to provide bipolar pacing. In addition, the lead structure 18 may include several individual leads, each of which may include multiple conductors, if desired. This provides for redundancy as will be discussed below. While FIG. 1 illustrates the lead structure 18 connected directly to the pulse generator 16, the lead structure would preferably be connected to one or more ports or terminals that are provided on a housing H that includes the power source 12, controller 14 and pulse generator 16. Where multiple leads are used, a separate terminal will typically be provided for connection to each lead. Similarly, where each lead includes multiple conductors, each terminal will provide for a separate connection for each of the conductors. The controller 14 provides control signals to the pulse generator 16 to indicate which pacing signals or information are to be provided to which terminal, or connector.

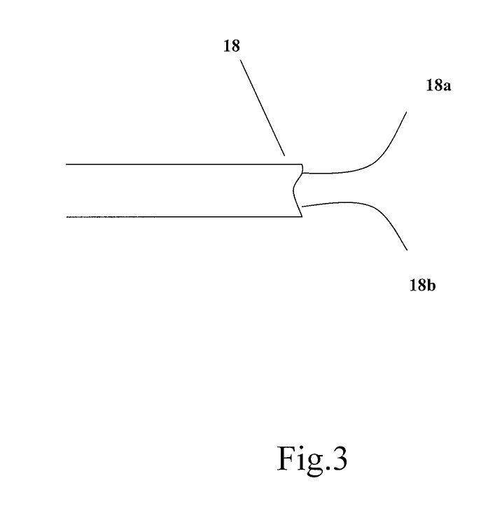

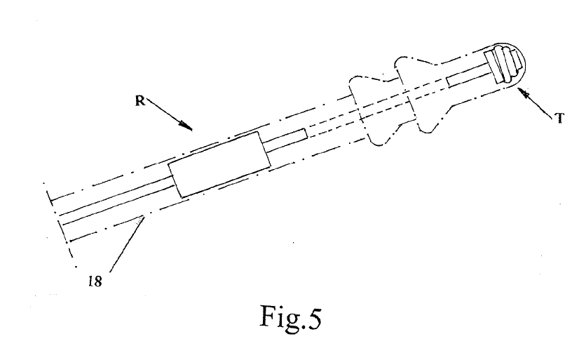

The lead structure 18, or the individual leads therein (see leads 18a, 18b for example of FIGS. 2-3) will typically include an electrode mounted on the distal end thereof. The electrode provides actual contact with the heart. In a bipolar lead, such as that illustrated in FIG. 5, two electrodes are provided. A tip electrode T is provided at the distal end and a ring electrode R is provided prior to the distal end and separated from the tip electrode by some distance. Both of these electrodes contact the heart itself at the desired location. Each electrode is connected to a separate conductor within the lead. There are various other types of electrodes that may be used in the pacing system 10, including, but not limited to passive fixation leads and active fixation leads. Further, it is not necessary to provide bipolar pacing. The system 10 may be used to provide unipolar pacing, if desired. In this case, a single electrode would typically be provided at the distal end of the lead and only a single conductor would be required.

As is noted above, failures in the lead, or leads, are common in conventional pacing systems. In a preferred embodiment, the lead structure 18 is configured to ease insertion and positioning of the electrodes in the heart and to provide for redundancy to reduce the risk of a total pacing failure. FIGS. 2-3 illustrate a preferred embodiment of the lead structure 18. Multiple leads 18a, 18b are provided in the structure 18 for redundancy. More specifically, the two leads 18a, 18b are provided in a unitary structure surrounded by a sugar moiety M, as illustrated in the cross-sectional view of FIG. 2. The unitary structure eases insertion of the leads 18a, 18b into the heart. Once in the heart, the sugar moiety M dissolves, as illustrated in FIG. 3, for example, to allow the leads 18a, 18b to separate for implantation at different locations in the heart. While two leads 18a, 18b are illustrated in the lead structure 18, additional leads may be provided in the unitary structure, if desired. The sugar moiety M may be selected such that it dissolves at a desired rate to maintain unity of the leads 18a, 18b for as long as is desired. In one example, the sugar moiety may be mannitol. Any suitable sugar moiety, however, may be used. It is preferred that the sugar moiety dissolve in the user's bloodstream during a time period of between 2 minutes and five minutes.

The leads 18a, 18b may provide redundancy on several levels. First, since the leads 18a,18b are implanted at different locations in the heart, if one lead becomes loose or otherwise ineffective, pacing signals may be provided to the other lead to continue proper pacing. Indeed, if one of the leads 18a, 18b fails altogether, the other lead may be used to provide pacing signals to the user's heart on a permanent basis. The location at which each lead 18a, 18b is connected to the heart is preferably selected to ensure that it is suitable for providing backup pacing when necessary or desired. Preferred locations are areas with good electrical signals such that they provide adequate pacing and sensing. In addition, one of the leads 18a, 18b may be used to provide sensed information regarding conditions or activity of the heart itself. This information may be used to ensure that the heart is responding properly to pacing signals and may also be used to provide fault detection. The same electrode may be used for sensing and pacing.

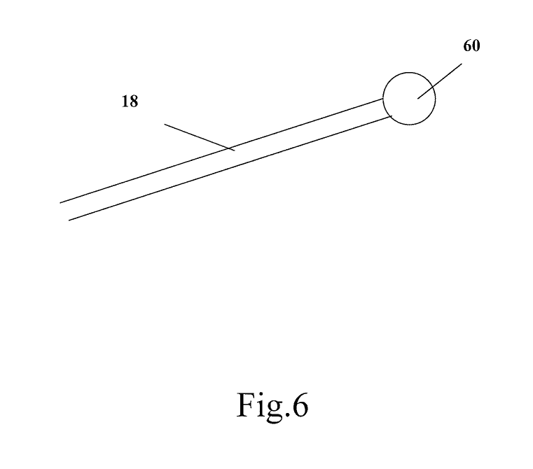

Specifically, in an embodiment, the unitary structure 18 is threaded into a vein and into the heart, typically the right ventricle. The sugar moiety M dissolves to allow the individual leads 18a, 18b to separate for implantation at desired locations in the heart. The lead 18a is used to provide pacing. That is, the pulse generator 16 provides pacing signals to the heart via the lead 18a, which are conveyed to the heart via electrodes, such as electrode 60 of FIG. 6, for example, positioned on the distal end of the lead. The second lead 18b, and the electrode positioned thereon, are used to sense conditions in the heart and convey this sensed information to the controller 14, via the generator 16, for example. That is, the lead 18b, and/or an electrode attached thereto, may be used to sense the evoked response of the heart to a pacing event stimulated by the lead 18a. If the heart does not respond, this information may be supplied to the controller 14, which may take action to correct or compensate, as will be described further.

It is preferred that the lead structure 18 is threaded into the vein via a sheath until it is positioned in a desired chamber of the heart. This prevents the possibility of the lead structure 18 getting stuck in proximal vasculature, which may result in premature dissolving of the sugar moiety M.

In the event that no evoked response is detected, the controller 14 may instruct the pulse generator 16 to provide the pacing signal via the second lead 18b. This may occur on a temporary basis, at least at first. After a period of time, however, the controller may continue for a period of time before pacing is attempted via the first lead again. Where an evoked response is sensed using the second lead, this lead is blanked, typically for a period of 20-30 milliseconds. During this period, no signal is transmitted to the second lead 18b and pacing continues via the first lead 18a. Conditions that may be sensed include a very high impedance, generally indicating that the lead or electrode has become dislodged and very low impedance, indicating some sort of short circuit. In either case, the sensed information may be used by the controller 14 to modify operation and ensure proper pacing. Only two leads 18a, 18b are necessary to provide redundancy, however, additional leads may be provided. Further, the two leads do not necessarily have to be positioned in the same chamber. One could be positioned in a left side chamber while the other may be provided in its right side counterpart, for example.

While the leads 18a, 18b are shown schematically as single conductors, each of the leads may include multiple conductors as mentioned above. In an embodiment, in the event of a fault in these leads or conductors, the controller 14 may revert to unipolar pacing. That is, pacing signals may be provided via a single conductor of the lead 18a, 18b which is still in effective contact with the heart. Further, in an embodiment, in the event of a fault in the controller 14 itself, the pulse generator 16 may include default circuitry to provide for a constant pulse signal of a desired frequency and amplitude to maintain pacing even where positive control has been lost.

Additional leads may be added to the lead structure 18 as desired to provide pacing and/or sensing in the same chamber or multiple chambers of the heart. The use of additional leads allows for redundancy in multiple chambers of the heart. Biventricular pacers, for example, may be provided which typically pace from the right ventricle and left ventricle and sense from the right ventricle. The pacing may be provided simultaneously or sequentially and preferably is provide to provide proper right ventricle/left ventricle delay to optimize heart output. Redundancy may be provided in both pacing and sensing, if desired, using additional leads in the structure 18, for example.

In one embodiment, the system 10 may be provided with redundancy on multiple levels. For example, the system 10 may include several power sources 12 and may allow for switching between power sources as an individual power source is drawn down or fails. Similarly, the system 10 may include multiple controllers 14 and/or multiple pulse generators 16, if desired. These redundant elements may be selectively utilized in the event of a failure.

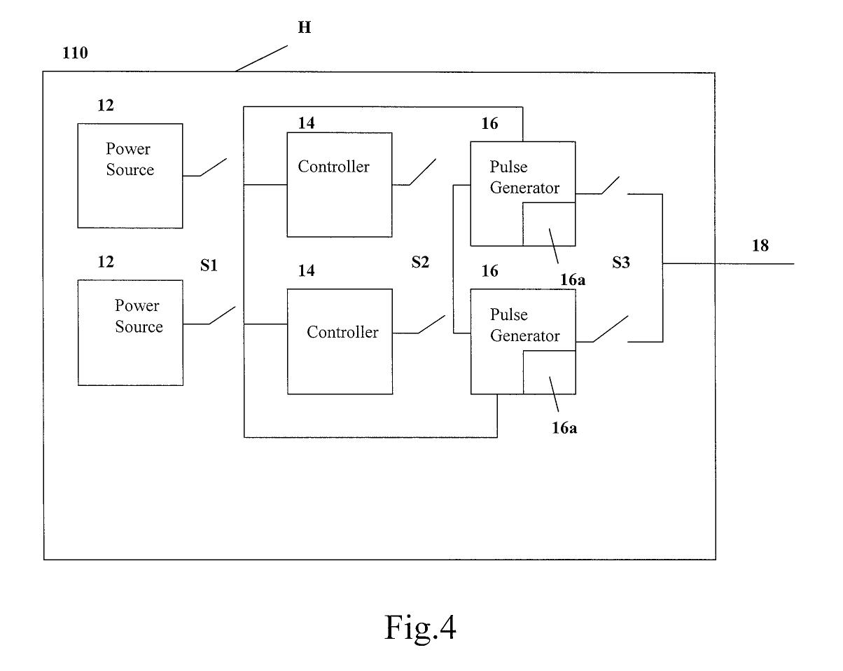

FIG. 4 illustrates an exemplary embodiment of a system 110 that includes such redundant components. Most simply, a pair of power sources 12 are provided and selectively connected to the other elements via the switches S1. In the event that one power source 12 fails, it can be disconnected and power may be provided by the second power source by operation of the switches S1. Similarly, two controllers 14 are provided and are selectively connectable to either of the two pulse generators 16 via the switches S2. Thus, if there is a fault in one of the controllers 14, the other controller may be used to control the pulse generator 16. The pulse generators 16 are, in turn, selectively connected to the lead structure 18 via the switches S3. In the event of a failure in one of the generators 16, the other generator may be used to provide pacing signals to the lead structure 18. If desired, additional redundant units may be provided, for example, three or four power sources, controllers and/or pulse generators may be used. While FIG. 4 illustrates three different pairs of switches S1, S2, S3 the device 110 may use fewer or additional switches, if desired. Generally, operation of the switches S1, S2, S3 will be controlled by one of the controllers 14, however, the switches may be operated remotely as well, if desired. As noted above, the lead structure 18 preferably already includes redundant leads 18a, 18b, and additional redundant leads may also be provided if desired, as suggested above.

In the event of any sort of fault, the system 10, 110 will preferably provide an alert signal to the user (patient) or overseeing doctor or medical team. For the former, the alert signal may be an audible alert or signal. In the latter case, the alert maybe in the form of a remote monitoring flag, page etc. This will allow the user and or his doctor to arrange for maintenance, repair or even replacement of the system, if necessary. Since the system 10, 110 is typically provided in the user's body, the alert signal is preferably transmitted wirelessly outside of the body to an external scanner or transceiver. Such devices are commonly used to communicate with implanted pacing systems to monitor usage and to provide for reprogramming if necessary.

In the above examples, at least one of the leads 18a, 18b maintains operation even if the other fails. If all of the leads fail, however, no pacing may be provided which could endanger the patient's life. Accordingly, the system 10, 110 of the present disclosure also provides for leadless, or wireless pacing.

In this mode, pacing signals are transmitted to desired sites in the heart wirelessly. As noted above, the pulse generator 16 preferably includes a transceiver 16a. This transceiver 16a transmits the pacing information wirelessly to electrodes implanted in the heart, preferably at the end of the leads 18, 18a, 18b. The electrodes then apply appropriate pacing stimulus to the heart based on the pacing signals transmitted by the transceiver 16a. In an embodiment, the transmission of the pacing information is accomplished by a radio frequency (RF) signal. Generally, transmission will occur within a range of about 3 kHz to 300 GHz, however, any suitable frequency may be used. In a preferred embodiment, the pacing information may be encrypted prior to transmission. Further, in an embodiment, a narrow medical frequency band may be defined and used for transmission of the pacing information, as well. Both encryption and transmission over a defined medical band will reduce interference and errors in the reception of the pacing information. While wireless transmission of pacing signals is preferably accomplished via RF, or other suitable electromagnetic transmission, any suitable wireless transmission medium may be used. Ultrasound, for example, may be used to transmit the pacing information and/or receive information as well, if desired. That is, the transceiver 16a may include an ultrasound transmitter and/or receiver.

The electrodes, such as electrode 60, for example, at the end of the leads 18a, 18b used in the system 10, 110 described above are positioned as desired in the user's heart. In ordinary operation, pacing signals from the pulse generator 16 are provided to the electrodes via the leads 18a, 18b and are applied to the heart. FIG. 6 illustrates a schematic view of a lead 18 with an electrode 60 mounted on a distal end thereof. As discussed above, the electrode 60 actually contacts the heart. The electrode 60 preferably also includes at least a receiving circuit to receive the pacing signals or information transmitted by the transceiver 16a. The electrode 60 of FIG. 6 is illustrated in schematic form and may take any of several different forms. The electrode 60 may also include a transmitting circuit as well, such that the electrode 60 includes a transceiver.

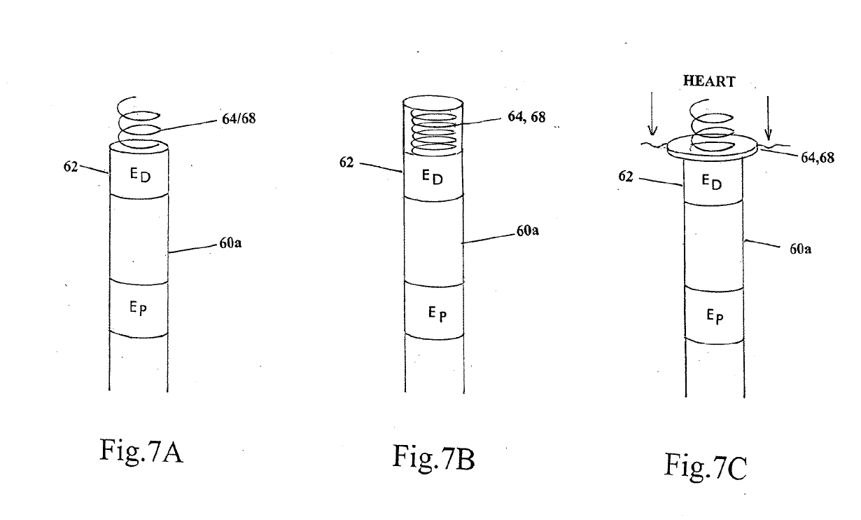

In one embodiment, a retained screw electrode 60a may be used as illustrated in detail in FIGS. 7A-7C. A typical active fixation lead includes a distal electrode 62. In one embodiment, an antenna 64, which may include appropriate receiving circuit 68 is provided in this distal electrode 62 to received pacing information. As the screw of the lead is advanced, and the lead is secured to the heart, the coiled antenna, which is initially cylindrical as shown in FIG. 7A, compresses against the heart as can be seen in FIG. 7B, for example. The lead torque allows the antenna to increase its radius as it is compressed against the heart. FIG. 7C shows the electrode 60a connected to the heart with the antenna 64 compressed and spread radially against the heart. Energy may be provided to the electrode 60a, preferably via RF as noted above. The energy, however, may be transmitted by magnetic induction or any other suitable electromagnetic transmission medium. The antenna 64 absorbs the energy of the transmission to drive the distal pacing electrode 62. That is, the transmission of the transceiver 16a induces a current in the antenna, which may be applied to the heart to provide the pacing stimulus. While an active fixation electrode 60a is illustrated in FIGS. 7A-7C, the system 10, 110 of the present application may utilize different electrodes, if desired, provided that they include a receiving circuit to receive transmissions from the transceiver 16a. When the lead 18 is operating normally, however, the electrode 60a operates as a conventional electrode. That is, the antenna 64 and receiving circuit 68 do not inhibit normal operation of the electrode 60a and pacing information from the lead drives the electrode. In the event of a fault, however, an alert will signal indicating: (1) a switch to the transceiver of the pulse generator 16 to provide wireless pacing; (2) an audible alert to advise the patient to seek immediate medical attention; and (3) an alert to the overseeing doctor or medical team identifying the fault is provided, so that it can be addressed in a timely manner. Also, a larger antenna may be housed in the pulse generator 16 for use by its transceiver 16a.

In the embodiment of FIGS. 7A-7C, the electrode 60a receives the pacing signals via RF or other electromagnetic transmission. In the event that the transceiver 16a utilizes an ultrasound transmitter, as discussed above, the receiving circuit of the electrode 60, 60a will include an ultrasound transducer operable to convert the acoustic signal transmitted by the ultrasound transmitter into electricity used to stimulate the user's heart.

Further, while the electrodes 60, 60a discussed above are illustrated as part of a lead 18, the electrodes 60, 60a may be implemented as independent elements. That is, the system 10, 110 may include electrodes that are mounted on the distal end of a lead 18 in a conventional manner that provide pacing and sensing functions as described above and communicate via the lead 18. In the event of a fault in the lead, or otherwise, the pulse generator 16 may switch to wireless transmission of pacing signals. These pacing signals may be received by the independent electrodes, which are similar in structure to electrodes 60, 60a and include receiving circuitry, but are separate from the lead 18. These electrodes are also positioned at desired locations within the user's heart. In this embodiment, the lead 18 and the electrode provided on the end thereof are similar to a conventional lead and electrode pairing. The independent electrodes are used in the event of a lead failure to receive the wirelessly transmitted pacing signals and provide pacing to the heart. While the electrodes 60, 60a are described above as including a receiving circuit, a transmitting circuit may also be included such that the electrodes 60, 60a include a transceiver to receive information and to send information. Sent information may include sensed information regarding conditions or activity in the heart, for example, as is discussed above.

Thus, the system 10, 110 preferably operates in at least two modes. During normal operation, pacing signals are provided to the heart via the lead structure 18. The lead structure 18 preferably includes multiple leads that provide redundancy and transmits pacing signals to the heart while also allowing for transmission of sensed information from the heart back to the pulse generator 16 or controller 14. In the event of a lead failure, that is, a failure of all leads, the controller 14 controls the transceiver 16a to transmit pacing information wirelessly. This information is received at the electrodes, such as electrode 60, positioned at the end of the lead or leads. The electrodes apply appropriate pacing signals to the heart to maintain pacing. As noted above, the electrode may alternatively be provided as a separate device from the lead 18.

The system 10, 110 of the present disclosure may operate in other modes as well. For example, as is discussed above, where a single lead fails, a second lead may be used to provide pacing signals, either temporarily or permanently. Further, while the system 10, 110 may be intended to provide bipolar pacing, in the event of a failure of one of the electrodes, unipolar pacing may be provided. In addition, there are several conventional therapies that require specific pacing modes. The system of the present application may be used in conjunction with any of these conventional pacing modes as well. Redundancy may be provided on multiple levels with redundant power sources, 12, redundant controllers 14, redundant pulse generators 16. Further, redundant leads 18a, 18b are preferably provided in a unitary structure to ease insertion into the heart and provide for later separation and attachment at different positions in the heart for redundancy. Redundancy may be provided for pacing and or sensing in one or more chambers of the heart and any suitable number of leads desired from such redundancy may be incorporated into the unitary structure described above.

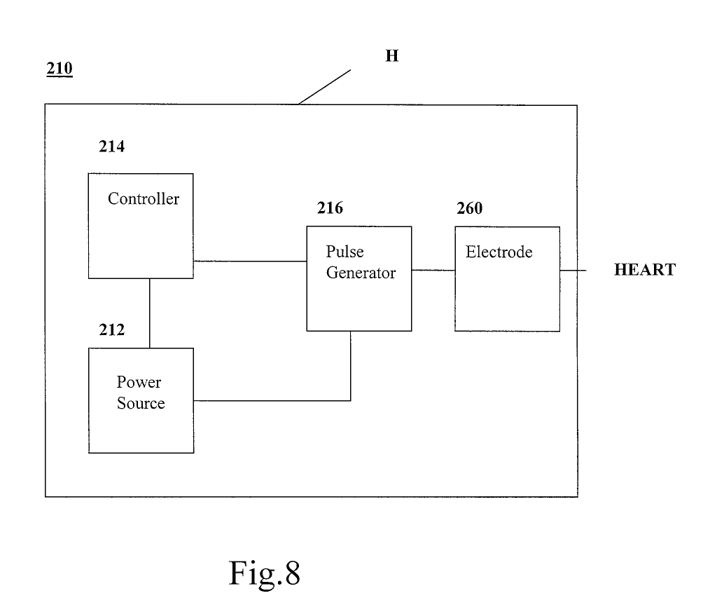

In another embodiment, illustrated in FIG. 8, for example, a pacing system 210 may include a power source 212, controller 214 and electrode 260 are positioned in housing H which is positioned in the heart. The controller 214 controls the pulse generator 216 to provide pacing signals in the manner described above. These pacing signals are provided to the electrode 260, which is also positioned within the housing H, and in contact with the user's heart. The electrode 260 stimulates the user's heart based on the pacing signals from the pulse generator 216 in a manner similar to that described above. That is, in the embodiment of FIG. 8, there is no need for any sort of external lead at all. The housing H is appropriately positioned in the heart such that the electrode 260 is in contact with the heart at a desired location. Additional housings H including similar components may also be provided at other positions in the heart as well. In a preferred embodiment the controller 214 or the pulse generator 216 are provided with a transceiver to allow wireless communication. This allows for redundancy similar to that described above.

One controller 214 may be used as a master controller for all other units, if desired and communicates wirelessly therewith, via transceiver 216a, for example. In addition, information may be transmitted and received to and from the exterior of the heart and user's body as well. The provision of multiple housings H, each including the elements described above, allows for redundancy. The master controller may control the various units such that some are used for primarily for pacing and others are used primarily for sensing. Pacing and sensing units may be controlled to switch functions, if desired or necessary, in the event of a fault or failure in one or more of the housings H. The master controller will preferably control operation of the components in each housing H as appropriate to provide constant pacing. The housing H may be introduced to the heart via a catheter. In an embodiment, the housing is attached to the heart via a screw-type fastener. Any other suitable fastener, however, may be used to secure the housing H to the desired position in the user's heart.

In an embodiment, a second electrode may be provided in the housing H and connected to the pulse generator 216 such that there are two contact points with the heart in the same general location. This provides another level of redundancy in the event of a failure at the electrode 260, for example. Additional electrodes may be added as well.

In an embodiment, the housing H described above may be itself mounted on the end of a lead, such as leads 18, 18a, 18b discussed above. That is, the housing H may act more or less as an electrode in that it will generally be used to stimulate the heart via the electrode 260 based on pacing signals that are provided from the lead 18 in the manner described above. These pacing signals may be provided directly to pulse generator 216 or via the controller 214 and then to the electrode 260. Further, as noted above, the pulse generator 216 and controller 214 may be embodied in a single device if desired. In the event of a lead failure, however, the controller 214 may continued to control the pulse generator 216 to provide desired pacing signals independently. In addition, an alert may be provided to alert the user and/or the monitoring medical team of the fault in the lead. In this embodiment, the controller 214 may be eliminated and the pulse generator 216 may be provided with simple instructions and/or circuitry to provide default pacing signals, as desired.

While the leads 18. 18a, 18b and electrodes 60, 60a discussed herein have been described as receiving pacing signals and providing sensing functions, they may also be used in conjunction with a defibrillation or other cardioversion system as well. In this case, the electrodes 60, 60a may detect a fibrillation in the heart. The controller 14 may control the pulse generator 16 to provide a defibrillation signal to the electrode 60, 60a. If there is a fault in the lead, the defibrillation signal may be transmitted wirelessly, or may be transmitted via a different lead to another electrode positioned in the heart, if desired. That is, redundancy via both leads and leadless systems may be provided for defibrillation as well as pacing.

Although the present invention has been described in relation to particular embodiments thereof, many other variations and modifications and other uses will become apparent to those skilled in the art.

What is claimed is:

1. A lead structure for use in a redundant pacing system comprising:
a first lead element including at least one conductor connected to a first terminal of a pulse generator of the pacing system;
a second lead element including at least a second conductor and connected to a second terminal of the pulse generator of the pacing system;
the first lead element and the second lead element held together via a sugar moiety for a predetermined period of time in a user's body.

2. The lead structure of claim 1, further comprising at least a third lead element connected to a third terminal of the pulse generator.

3. The lead structure of claim 1, wherein the sugar moiety comprises mannitol.

4. The lead structure of claim 1, wherein the sugar moiety is made of a material that dissolves two minutes to five minutes after contact with the user's blood stream.

5. The lead structure of claim 1, wherein the first lead element and the second lead element include at least two conductors.

6. The lead structure of claim 4, wherein the first lead element and the second lead element, respectively, include a first bipolar electrode and a second bipolar electrode positioned on a distal end thereof.

7. The lead structure of claim 1, wherein the first lead structure and the second lead structure, respectively, include a first and a second electrode positioned at the distal end thereof.

8. The lead structure of claim 1, further comprising a removable sheath surrounding the first lead element, the second lead element and the sugar moiety and operable for removal after the first and second lead elements are positioned in the user's heart.

9. A pacing system comprises:
a controller operable to provide control signals indicating desired pacing signals for use in stimulating a user's heart;
a pulse generator connected to the controller and operable to receive the control signals and to generate the desired pacing signals based on the control signals;
at least one lead electrically connected to the pulse generator and extending into the user's heart and operable to provide the pacing signals to the user's heart;
at least one electrode positioned in the user's heart and electrically connected to the at least one lead, the at least one electrode in contact with the user's heart and operable to stimulate the heart based on the pacing signals; and
a transceiver, in communication with the pulse generator and operable to selectively transmit the pacing signals to the electrode wirelessly;
wherein the transceiver is controlled by the controller to transmit the pacing signals when pacing signals are not received by the electrode from the at least one lead.

10. The pacing system of claim 9, wherein the electrode includes a receiving circuit operable to receive the pacing signals selectively transmitted by the transceiver.

11. The pacing system of claim 10, wherein the receiving circuit includes an antenna in which an electrical current is induced by the transmitted pacing signals and applied to the heart.

12. The pacing system of claim 9, wherein the controller controls the transceiver to transmit the pacing signals at a predetermined frequency after a fault is detected in the lead.

13. The pacing system of claim 12, where in the predetermined frequency is a radio frequency.

14. The pacing system of claim 13, wherein the radio frequency is in a range of 3 kHz to 300 GHz.

15. The pacing system of claim 12, wherein the pacing signals are encrypted for transmission by the transceiver to minimize interference.

16. The pacing system of claim 12, wherein the predetermined frequency is a frequency suitable for inducing a current in a conductor.

17. The pacing system of claim 10, wherein the transceiver further comprises an ultrasound transmitter and the controller controls the transceiver to transmit the pacing signals using the ultrasound transmitter.

18. The pacing system of claim 17, wherein the receiving circuit includes an ultrasound transducer operable to receive the pacing signals transmitted by the ultrasound transmitter and provide electrical stimulation to the user's heart based on the pacing signals.

19. The pacing system of claim 9, wherein the at least one lead comprises:
a first lead element including at least one conductor connected to a first terminal of the pulse generator;
a second lead element including at least a second conductor and connected to a second terminal of the pulse generator;
the first lead element and the second lead element held together via a sugar moiety for a predetermined period of time in the user's body.

20. The pacing system of claim 9, further comprising a second lead electrically connected to the pulse generator and extending into the user's heart and operable to sense conditions in the user's heart and provide sensed information related to conditions in the users heart to the pulse generator.

21. The pacing system of claim 20, wherein pulse generator conveys the sensed information to the controller and the controller generates the control signals based on the sensed information.

22. The pacing system of claim 20, wherein the second lead is further operable to provide pacing in the user's heart based on pacing signals provided by the pulse generator.

23. A pacing system comprises:
a controller operable to provide control signals indicating desired pacing signals to stimulate a user's heart;
a pulse generator connected to the controller and operable to receive the control signals and to generate the desired pacing signals based on the control signals;
at least one lead electrically connected to the pulse generator and extending into the user's heart and operable to provide the pacing signals to the user's heart;
at least a first electrode positioned in the user's heart and electrically connected to the at least one lead, the first electrode in contact with the user's heart and operable to stimulate the heart based on the pacing signals;
a transceiver, in communication with the pulse generator and operable to selectively transmit the pacing signals wirelessly; and
a second electrode separate from the lead and positioned in the user's heart, the second electrode including a receiving circuit operable to receive the wireless pacing signals and operable to stimulate the user's heart based on the received wireless pacing signals,
wherein the transceiver is controlled by the controller to wirelessly transmit the pacing signals when pacing signals are not received by the first electrode from the at least one lead.

24. The pacing system of claim 23, wherein the receiving circuit includes an antenna in which an electrical current is induced by the transmitted pacing signals and applied to the heart.

25. The pacing system of claim 23, wherein the controller controls the transceiver to transmit the pacing signals wirelessly at a predetermined frequency after a fault is detected in the lead.

26. The pacing system of claim 25, where in the predetermined frequency is a radio frequency.

27. The pacing system of claim 26, wherein the radio frequency is in a range of 3 kHz to 300 GHz.

28. The pacing system of claim 25, wherein the pacing signals are encrypted for transmission by the transceiver to minimize interference.

29. The pacing system of claim 25, wherein the predetermined frequency is a frequency suitable for inducing a current in a conductor.

30. The pacing system of claim 23, wherein the transceiver further comprises an ultrasound transmitter and the controller controls the transceiver to transmit the pacing signals using the ultrasound transmitter.

31. The pacing system of claim 30, wherein the receiving circuit includes an ultrasound transducer operable to receive the pacing signals transmitted by the ultrasound transmitter and provide electrical stimulation to the user's heart based on the pacing signals.

32. The pacing system of claim 23, wherein the at least one lead comprises:
a first lead element including at least one conductor connected to a first terminal of the pulse generator;
a second lead element including at least a second conductor and connected to a second terminal of the pulse generator;
the first lead element and the second lead element held together via a sugar moiety for a predetermined period of time in the user's body.

33. The pacing system of claim 23, further comprising a second lead electrically connected to the pulse generator and extending into a user's heart and operable to sense conditions in the user's heart and provide sensed information related to conditions in the users heart to the pulse generator.

34. The pacing system of claim 33, wherein pulse generator conveys the sensed information to the controller and the controller generates the control signals based on the sensed information.

35. The pacing system of claim 33, wherein the second lead is further operable to provide pacing in the user's heart based on pacing signals provided by the pulse generator.

36. A pacing system comprises:
a housing configured for positioning in a user's heart;
a controller, mounted in the housing and operable to provide control signals indicating desired pacing signals for use in stimulating the user's heart;
a pulse generator, mounted in the housing and connected to the controller and operable to receive the control signals and to generate the desired pacing signals based on the control signals;
at least a first electrode, mounted in the housing and electrically connected to the pulse generator, the first electrode in contact with the user's heart and operable to stimulate the heart based on the pacing signals; and
a fastener configured and operable to attach the housing to the user's heart such that the electrode is in contact with the user's heart.

37. The pacing system of claim 36, further comprising a second electrode mounted in the housing and electrically connected to the pulse generator, the second electrode in contact with the user's heart and operable to stimulate the heart based on the pacing signals when a fault is detected in the first electrode.

38. The pacing system of claim 36, further comprising:
a transceiver connected to the controller and mounted in the housing, the transceiver operable to transmit control signals from the controller out of the housing wirelessly;
a second housing configured for positioning in a user's heart;
a second transceiver mounted in the second housing configured and operable to receive at least the control signals transmitted by the transceiver;
a second controller connected to the second transceiver and mounted in the second housing, the second controller configured and operable to provide second control signals indicating desired pacing signals for use in stimulating the user's heart;
a second pulse generator, mounted in the second housing and connected to the second controller and operable to receive the second control signals and to generate the desired pacing signals based on the second control signals;
at least a second electrode, mounted in the second housing and electrically connected to the pulse generator, the second electrode in contact with the user's heart and operable to stimulate the heart based on the desired pacing signals; and
a second fastener configured and operable to attach the second housing to the user's heart such that the second electrode is in contact with the user's heart,
wherein the second controller provides the second control signals based on received control signals from the transceiver.

39. The pacing system of claim 36, further comprising a lead connected to the housing and electrically connected to at least the controller and the control signals provided by the controller are based on instructions provided via the lead.

40. A pacing system comprising:
a controller operable to provide control signals indicating desired pacing signals for use in stimulating a user's heart;
a pulse generator connected to the controller and operable to receive the control signals and to generate the desired pacing signals based on the control signals;
a first lead electrically connected to the pulse generator and extending into the user's heart to a first position;
a first electrode positioned in the user's heart at the first position and electrically connected to the first lead, the first electrode configured and operable to stimulate the user's heart based on the pacing signals from the pulse generator and to sense activity in the first position in the user's heart and to provide first sensed information regarding the activity in the first position to the pulse generator and controller;
a second lead electrically connected to the pulse generator and extending into the user's heart to a second position;
a second electrode positioned in the user's heart at the second position and electrically connected to the second lead, the second electrode configured and operable to stimulate the user's heart based on the pacing signals and to sense activity at the second position in the user's heart and to provide second sensed information regarding the activity at the second position to the pulse generator;
wherein the controller controls the pulse generator to provide pacing signals to the first electrode via the first lead for a period of time and to provide pacing signals to the second electrode via the second lead when the first sensed information indicates a fault in one of the first lead and the first electrode.

41. The pacing system of claim 1, wherein the pulse generator further provides defibrillation signals based on the control signals and wherein the defibrillation signals are sent to the first electrode via the first lead for a period of time and to the second electrode via the second lead when the first sensed information indicates a fault in one of the first lead and the first electrode.
